(19) United States
(12) Patent Application Publication
(10) Pub. No.: US 2025/0264715 A1
(43) Pub. Date: Aug. 21, 2025

(54) SYSTEM, APPARATUS, METHOD AND COMPUTER-ACCESSIBLE MEDIUM FOR CONTROLLING A GENERATION OR AN OUTPUT OF AN ELECTROMAGNETIC RADIATION

(71) Applicant: SPECTRAWAVE, Bedford, MA (US)

(72) Inventors: Eman NAMATI, Concord, MA (US); Peter VOKROT, Malden, MA (US); Tsung-Han TSAI, Newton, MA (US); Barry VUONG, Waltham, MA (US); Damon DEPAOLI, Arlington, MA (US)

(21) Appl. No.: 19/196,532

(22) Filed: May 1, 2025

Related U.S. Application Data
(63) Continuation of application No. PCT/US2023/036569, filed on Nov. 1, 2023.
(60) Provisional application No. 63/421,351, filed on Nov. 1, 2022.

(51) Int. Cl. G02B 26/10; G01D 5/347

(52) U.S. Cl. CPC G02B 26/101; G01D 5/3473

(57) ABSTRACT

Exemplary apparatus, system, method and computer-accessible medium for controlling a generation or an output of an electromagnetic radiation can be provided. For example, with a device, directing the electromagnetic radiation, and using a controller, controlling the device to move the electromagnetic radiation at a rate of motion. The device and/or the controller can provide information regarding the rate of motion. Further, with a computer, it is possible to receive the information, and effectuate the generation or the output of the electromagnetic radiation if the rate of motion is greater than a predetermined rate based on the information.

### CROSS-REFERENCE TO RELATED APPLICATION(S)

This application relates to and claims priority from U.S. Patent Application No. 63/421,351, filed on Nov. 1, 2022, the entire disclosure of which is incorporated herein by reference.

### FIELD OF THE DISCLOSURE

The present disclosure relates generally to the technology of electromagnetic radiation, and more particularly, to system, apparatus, method and computer-accessible medium for controlling a generation or an output of an electromagnetic radiation.

### BACKGROUND INFORMATION

Electromagnetic radiation can cause damage to various degrees. For example, exposure to high levels of electromagnetic radiation can cause acute health effects such as skin burns and acute radiation syndrome. As an example of electromagnetic radiation, laser exposures of eyes can have a wide range of effects including pain, irritation, headache, flash blindness, dazzle, dark spots, hazy vision, floaters, burns, retinal bleeding, and so forth.

Accordingly, there is a need to provide apparatuses, systems and/or methods to prevent or reduce electromagnetic radiation.

### SUMMARY OF EXEMPLARY EMBODIMENTS

Such issues and/or deficiencies can at least be partially addressed and/or overcome by providing system, apparatus and method for controlling a generation or an output of an electromagnetic radiation by providing exemplary embodiments according to the present disclosure.

According to an exemplary embodiment of the present disclosure, exemplary system, apparatus, method and computer-accessible medium for controlling a generation or an output of an electromagnetic radiation can be provided. For example, with a device, directing the electromagnetic radiation, and using a controller, controlling the device to move the electromagnetic radiation at a rate of motion. The device and/or the controller can provide information regarding the rate of motion. Further, with a computer, it is possible to receive the information, and effectuate the generation or the output of the electromagnetic radiation if the rate of motion is greater than a predetermined rate based on the information.

For example, a light source can be provided, and the generation or the output of the electromagnetic radiation can be provided via the computer by controlling the light source. It is also possible to control a frequency and/or an intensity of the electromagnetic radiation based on the information, e.g., using the computer. The device can include at least one component configured for the electromagnetic radiation to allow to pass or at least partially prevent from passing in a controlled manner, and it is possible to control the generation or the output of the electromagnetic radiation by controlling the at least one component. The component(s) can include a shutter or a light blocking configuration which can be configured to be opened or closed by the computer.

In another exemplary embodiment of the present disclosure, the device can include at least one optical component and a motor which can be configured to move the optical component(s). The movement of the optical component by the motor can be controlled by the controller. The controller can be provided in a catheter. The device can be configured to direct the electromagnetic radiation based on an optical modality (e.g., an optical coherence tomography modality). The controller can include a spatial encoder which can provide a trigger signal which can be associated with the information. The spatial encoder can be configured to detect the rate of motion.

According to still another exemplary embodiment of the present disclosure, the controller can include a catheter-lock switch which can provide a trigger signal that can be associated with the information. The catheter-lock switch can be configured to determine an installation status of a catheter. The controller can include an aperture position switch which can provide a trigger signal which is associated with the information. The aperture position switch can be configured to determine a position of a light aperture. In addition or alternatively, the controller can include an interlock circuit that can be configured to receive an input signal and transmit an output signal to the computer. The interlock circuit can be configured to control an intensity of the output of the electromagnetic radiation.

In a still exemplary embodiment of the present disclosure, the controller can include a temperature sensor to monitor a temperature associated with the rate of motion. The exemplary device can include a motor to direct the electromagnetic radiation.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a flow diagram of a method/process for preventing or reducing exposure of an electromagnetic radiation (e.g., a laser light source) according to an exemplary embodiment of the present disclosure;

FIG. 2 are a set of exemplary graphs illustrating various parameters used, relied on, obtained, etc. used by the exemplary methods/processes, systems and apparatus for preventing or reducing exposure of the electromagnetic radiation according to an exemplary embodiment of the present disclosure;

FIG. 3 is a diagram of a first exemplary apparatus/system using or in which the exemplary method/process illustrated in FIG. 1 can be implemented according to an exemplary embodiment of the present disclosure with a circular and/or helical light exposure;

FIG. 4 is a diagram of a second exemplary apparatus/system using or in which the exemplary method/process illustrated in FIG. 1 can be implemented according to another exemplary embodiment of the present disclosure with a raster motion/scan light exposure;

FIG. 5 is a diagram showing a third exemplary apparatus/system using or in which the exemplary method/process illustrated in FIG. 1 can be implemented according to yet exemplary embodiment of the present disclosure, which can include optical coherence tomography (OCT) components;

FIG. 6 is a diagram showing an apparatus/system having a safety feature for preventing or reducing the electromagnetic radiation exposure according to an exemplary embodiment of the present disclosure;

FIG. 7 is a diagram showing another exemplary apparatus/system having the safety feature for preventing or reducing the electromagnetic radiation exposure according to another exemplary embodiment of the present disclosure;

FIG. 8 is a diagram showing a multimodal characterization apparatus/system which includes a multimodal characterization system for coronary artery imaging according to yet another exemplary embodiment of the present disclosure;

FIG. 9 is a logic/block diagram of an exemplary system/apparatus which includes the safety feature for preventing or reducing the electromagnetic radiation exposure according to an exemplary embodiment of the present disclosure; and

FIG. 10 is a block diagram showing still another imaging apparatus/system having the safety feature for preventing or reducing electromagnetic radiation exposure according to still another exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the certain exemplary embodiments illustrated in the figures and the appended claims.

### DETAILED DESCRIPTION OF EXEMPLARY EMBODIMENTS

The following description of embodiments provides non-limiting representative examples referencing numerals to particularly describe features and teachings of different aspects of the present disclosure. The embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the invention to such an extent that other implementations, not specifically covered but within the knowledge of a person of skill in the art having read the description of embodiments, would be understood to be consistent with an application of the present disclosure.

Exemplary embodiments of exemplary system, apparatus, method and/or device of the present disclosure can be provided that can includes and/or implements a safety circuit for an electromagnetic radiation (e.g., light) energy hazard reduction. For example, in the exemplary embodiments of the present disclosure, the exemplary reduction of the laser light energy can be achieved to a degree where exposure does not pose a hazard to human eyes or skin by controlling and/or enforcing a specific (e.g., minimum) laser rotation speed or minimum accessible laser aperture distance, to decrease the effective laser safety classification (e.g., Class 1M). The corresponding functionality can be implemented using discrete electronic components, which can obviate the need for providing and/or developing additional software and the associated qualification and verification.

According to an exemplary embodiment of the present disclosure, the safety circuit for electromagnetic radiation (e.g., light) energy hazard reduction can be or include logic circuits in an arrangement that uses a low-frequency crystal oscillator as a time base to synchronize gates, discrete logic integrated circuits (ICs), transistors, and opto-isolator outputs. The hardware inputs to the safety circuit can include, for example, incremental encoder pulses of a rotation motor that spins the laser output aperture (e.g., rotation motor incremental encoder signals), catheter insertion detection sensor/switch signals, and laser “safe position” sensor signals (e.g., laser “safe position” optical switch signals).

According to an exemplary embodiment of the present disclosure, the safety circuit can include, e.g., an opto-isolator at the safety circuit output that provides galvanic isolation between the logic circuit and the laser. Additionally, digital outputs can be provided from the logic circuit that can be used for monitoring of the status of an interlock signal by, for example, a microcontroller. This signal can be, e.g., intentionally read-only, reduce or to eliminate the possibility of a faulty microcontroller from altering the interlock signal.

According to an exemplary embodiment of the present disclosure, pulses from the rotation motor encoder can be used to clock a shift register. The shift register can be, e.g., periodically reset at a fixed rate, and chosen to enforce a minimum rotation speed (or laser beam travel speed). If the rotation speed is sufficiently high, then the most significant bit of the shift register can likely be high and can be latched to facilitate the interlock (e.g., a safety circuit included in the interlock) to be de-asserted (e.g., laser on), given the state of catheter insertion detection circuit and/or laser “safe position” sensor. In some exemplary embodiments, to prevent/reduce unwanted oscillation at or around the minimum rotation speed set point, an exemplary circuit that provides digital hysteresis can be employed.

According to an exemplary embodiment of the present disclosure, the exemplary safety circuit can be implemented in several different ways. For example, the safety circuit can be integrated into an application-specific integrated circuit (ASIC) chip, which is a single chip solution rather than using several discrete logic chips. Alternatively, the safety circuit can be implemented in a field-programmable gate array (FPGA), a complex programmable logic device (CPLD), or other programmable logic device. As another option, the safety circuit, according to exemplary embodiments, can utilize alternative logic chips to optimize power consumption or to change logic voltage levels (e.g. 1.8 VDC, 5.0 VDC, etc.). As yet a further option, the safety circuit can utilize a different oscillator frequency, to change the rotation speed threshold if, for instance, a lower or higher laser power is used, or if laser safety standards change. As still another example according to exemplary embodiments, the safety circuit can have an additional/alternative output stage (opto-isolator or relay) to directly control the power supply of the laser source. Additionally, the safety circuit can be used for hazard reduction of an energy source other than laser light, e.g., radio frequency (RF), where rotation of the output aperture can effectively reduce incident power.

In another exemplary embodiment of the present disclosure, the safety circuit can be modified to add functionality, for example, by using an adjustable-frequency oscillator to allow different rotational speed setpoints, and/or adding more laser “safe position” sensors for different energy intensities.

According to a further exemplary embodiment of the present disclosure, the safety circuit can be purely hardware-based (i.e., no software). The safety circuit can use encoder pulses directly from existing motors to measure that a speed (e.g. rotational speed) is above the calculated “safety threshold” (e.g. a minimum revolution per minute (RPM) “safety threshold”), that is, the interlock can be controlled by pre-existing motor encoder signals. As used herein, the rotational/translational speed can also be referred to as and/or include a laser beam travel/motion speed (e.g. over a sample). The safety circuit of exemplary embodiments can incorporate a hardware-based “digital hysteresis” to prevent toggling at or near the threshold transition point. In this exemplary design, the output aperture of the laser can be open to the atmosphere and does not require a mechanical shutter (or similar) for added safety.

An exemplary electromagnetic radiation apparatus/system can be configured to control a generation or an output of an electromagnetic radiation. The electromagnetic radiation apparatus/system can comprise, e.g., a device configured to direct the electromagnetic radiation, and a controller configured to control the device to move the electromagnetic radiation at a rate of motion. The device and/or the controller can provide information regarding the rate of motion. Further, a computer can be provided which can be configured to receive the information, and effectuate the generation or the output of the electromagnetic radiation if the rate of motion is greater than a predetermined rate based on the information. Further, according to exemplary embodiments of the present disclosure, the computer can be configured to receive a first information signal and a second information signal and effectuate the generation or the output of the electromagnetic radiation if a first rate of motion and a second rate of motion (e.g., and/or a third rate of motion) are both greater than predetermined rates based on the first and second information signals. The electromagnetic radiation apparatus/system can further comprise a light source, whereas the computer can be configured to control the generation or the output of the electromagnetic radiation by controlling the light source. The computer can be configured to control a frequency and/or an intensity of the electromagnetic radiation based on the information. The device can include at least one component which can be configured to facilitate the electromagnetic radiation to pass or at least partially prevent from passing in a controlled manner. The computer can be configured to control the generation or the output of the electromagnetic radiation by controlling the at least one component. The component(s) can include a shutter or a light blocking configuration which can be configured to be opened or closed by the computer. The device can include at least one optical component and a motor which can be configured to move the at least one optical component. The movement of the optical component(s) by the motor can be controlled by the controller, which can be provided in, e.g., a catheter system. The device can be configured to direct the electromagnetic radiation based on an optical modality.

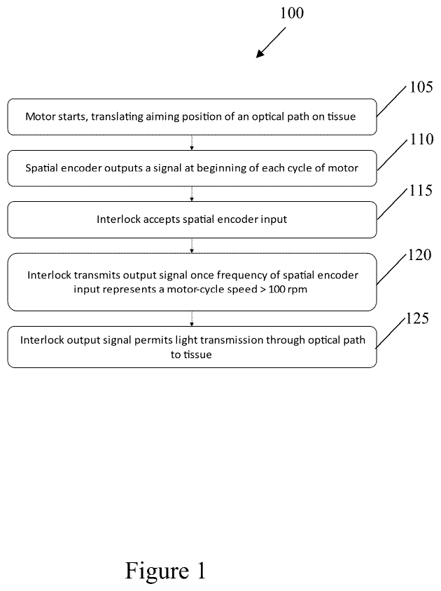

FIG. 1 shows a flow diagram of an exemplary method/process 100 for preventing or reducing exposure of an electromagnetic radiation (e.g., a laser light source) according to an exemplary embodiment of the present disclosure. The method/process 100 can be implemented in or using the exemplary electromagnetic radiation apparatus/system which can include a safety circuit, as described in various exemplary embodiments herein. As shown in FIG. 1, in step 105 of the method/process 100, the motor of the electromagnetic radiation can begin (or continue) translating an aiming position of an optical path on tissue. This can be done by adjusting the position, angle, etc. of various optical components and/or itself. In step 110, the spatial encoder associated with the motor can output a signal at beginning of each cycle of the motor. Each cycle of the motor can refer to a motion cycle of the motor. For example, the motor can rotate a cycle, and the motor can also move back and forth to complete a cycle (e.g., a reciprocal motion). In step 115, the interlock mechanism (which can include the safety circuit in the electromagnetic radiation system) can accept/receive the signal outputted by the spatial encoder (i.e., spatial encoder input). In step 120, the interlock mechanism can transmit an output signal when the frequency of spatial encoder input represents a motor-cycle speed equal to or greater than a predetermined motor-cycle speed, for example, 100 RPM. In step 125, the output signal transmitted by the interlock mechanism can permit light transmission through the optical path to the tissue. For example, when the frequency of spatial encoder input represents a motor-cycle speed smaller than the predetermined motor-cycle speed, the output signal transmitted by the interlock mechanism can completely or partially block light transmission through the optical path to the tissue, whereby preventing or reducing tissue damages caused by exposing to the light transmission.

In some exemplary embodiments, the motor-cycle speed can be other motor speeds rather than RPM, for example, in a laser raster scanning system/apparatus, can be a rate of movement across the tissue. According to some exemplary embodiments, the motor can oscillate, e.g., with one motion cycle of the motor can be a cycle of moving back and forth. Further, in some exemplary embodiments, a cycle can be defined as a cycle of doubling one rotation/oscillation of the motor, half of one rotation/oscillation of the motor, and so forth.

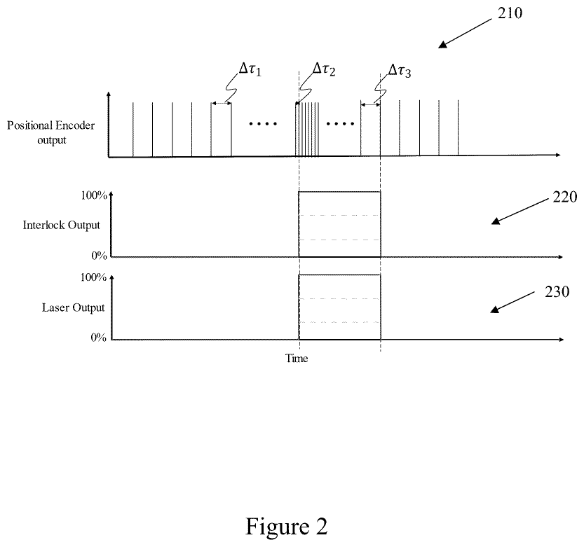

FIG. 2 illustrate a set of exemplary graphs illustrating various parameters used, relied on, obtained, etc. used by the exemplary methods/processes, systems and apparatus for preventing or reducing exposure of the electromagnetic radiation (e.g., a laser light source) according to an exemplary embodiment of the present disclosure. The steps being performed can correspond to those performed in the exemplary method/process 100 shown in FIG. 1. The graph 210 provides spatial/positional encoder output signals associated with the motor of the electromagnetic radiation system/apparatus versus time. The graph 220 shows exemplary interlock output signals of the interlock mechanism corresponding to the diagram 210 versus time. The graph 230 shows the light/energy output of the electromagnetic radiation system/apparatus corresponding to the graphs 210 and the graph 220. In this exemplary embodiment, the electromagnetic radiation system/apparatus is a laser system/apparatus. As shown in graphs 210, 220 and 230, when the frequency (e.g., the reciprocal of the motional cycle Δτ₁) of the spatial/positional encoder output signals associated with the motor of the laser system/apparatus is smaller than a specified threshold frequency, such as, e.g., smaller than about 10, 50, 100 RPM and everything in between, the interlock mechanism outputs a first signal to the controller of the laser system to block transmission of laser light along the optical path, such that the laser output is reduced, e.g., to zero energy output. For example, the outputted first signal can close a gate, such as a gate in the safety circuit, to block, prevent or reduce at least some form of light transmission whether the light transmission is through the interlock or not.

However, when the frequency (e.g., the reciprocal of the motional cycle Δτ₂) of the spatial/positional encoder output signals associated with the motor of the laser system/apparatus is greater than or equal to the specified threshold frequency, such as, e.g. greater than about 10, 50, 100, 1000, 10000, 100000 RPM and everything in between, the interlock mechanism can output a second signal to the controller of the laser system to facilitate the transmission of laser light along the optical path, such that the laser output is increased and/or maintained, e.g., to 100% energy output. For example, the outputted second signal can open a gate, such as, e.g., a gate in the safety circuit, to facilitate at least some light transmission, irrespective of whether the light transmission is through the interlock or not. Furthermore, as soon as the frequency (e.g., the reciprocal of the motional cycle Δτ₃) of the spatial/positional encoder output signals associated with the motor of the laser system/apparatus is once again less than or equal to the specified threshold frequency e.g., smaller than about 10, 50, 100 RPM and everything in between and beyond, the interlock mechanism outputs the first signal to the controller of the laser system to re-block transmission of laser light along the optical path, such that the laser output is reduced, e.g., to zero energy output.

Exposure to electromagnetic radiation (e.g., optical radiation) can cause harm to biological structures, due to thermal and non-thermal effects (e.g., photochemical). Careful calculation of electromagnetic radiation doses is critical for the safe use of light sources (e.g., lasers) clinically. The exemplary threshold frequency can be any suitable threshold frequency designed to reduce electromagnetic radiation to a safe range, considering laser characteristics (e.g., optical power, wavelength, repetition rate, pulse duration) and/or aspects of the experimental procedure (e.g., biological structure, beam spot size on sample, duration of exposure). The exemplary threshold frequency may, therefore, be calculated based on a known safety exposure limit (e.g., maximum permissible exposure (MPE), e.g., Nominal Ocular Hazard Distance (NOHD), e.g., Nominal Hazard Zone (NHZ)) designated by a classification document (e.g., IEC 60825, e.g., ANSI Z136).

For example, a frequency may be calculated in terms of RPM for a motor, (e.g., 10, 50, 100, 1000, 10000, 100000 RPM and everything in between and so forth) or it may be calculated in terms of Hertz of an imaging systems scanning speed (e.g., 0.167, 1.67, 16.7, 167, 1667 Hz and everything in between, and so forth). Conversely, a limiting motional cycle (e.g., Δτ₁ or Δτ₂ or Δτ₃, e.g., the reciprocal of the frequency) could be used to calculate a motor or scanning speed, e.g., being derived by a positional dwell time (e.g., pixel dwell time, e.g., voxel dwell time, e.g., irradiance dwell time on a biological structure), for example 1 us (e.g., 1 ps, 1 ns, 1 μs, 1 ms, 10 s and everything in between or beyond.

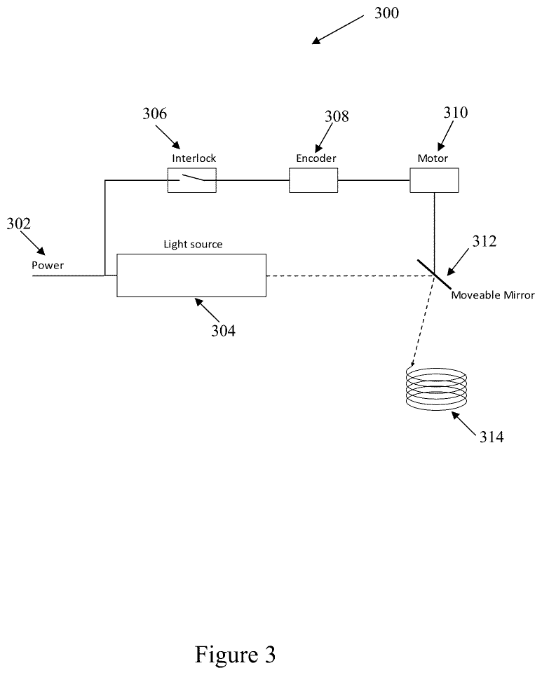

FIG. 3 shows a diagram of an exemplary apparatus/system 300 which can utilize the exemplary method/process illustrated in FIG. 1, according to an exemplary embodiment of the present disclosure. The exemplary apparatus/system 300 can be or include an electromagnetic radiation system such as a laser system. The system 300 can include a power supply 302, a light source 304 (such as a laser generation source), an interlock mechanism 306 including a safety circuit disclosed herein, a spatial encoder 308, a motor 310, and a moveable mirror 312. The interlock mechanism 306 can be configured to control the light transmission by either blocking or allowing the light transmission. The spatial encoder 308 can encode the motion cycle of the motor 310. The interlock mechanism 306 can receive signals/pulses from the spatial encoder 308, and can control the light transmission based on the received signals/pulses, as described herein. In this exemplary embodiment, the motor 310 can be configured to control the moveable mirror 312 to obtain a circular or helical trace of a light exposure 314 (e.g., the motor 312 performing rotational motion/scanning). The moveable mirror 312 can include a light shutter.

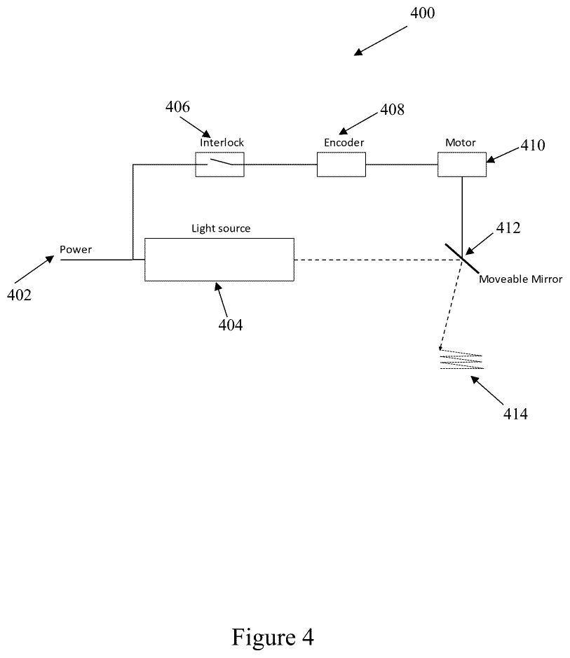

FIG. 4 shows a diagram of a second exemplary apparatus/system 400 which can utilize the exemplary method/process illustrated in FIG. 1, according to another exemplary embodiment of the present disclosure. The exemplary apparatus/system 400 can be or include an electromagnetic radiation system, such as, e.g., a laser system. The exemplary apparatus/system 400 can include a power supply 402, a light source 404 (such as a laser generation source), an interlock mechanism 406 including a safety circuit disclosed herein, a spatial encoder 408, a motor 410, and a moveable mirror 412. The interlock mechanism 406 can be configured to control the light transmission by either blocking or allowing the light transmission. The spatial encoder 408 can encode the motion cycle of the motor 410. The interlock mechanism 406 can receive signals/pulses from the spatial encoder 408, and can control the light transmission based on the received signals/pulses, as described herein. In this exemplary embodiment, the motor 410 can be configured to control the moveable mirror 412 to obtain trace of a light exposure 414 (e.g., the motor 412 performing raster motion/scanning). The moveable mirror 412 can include a light shutter.

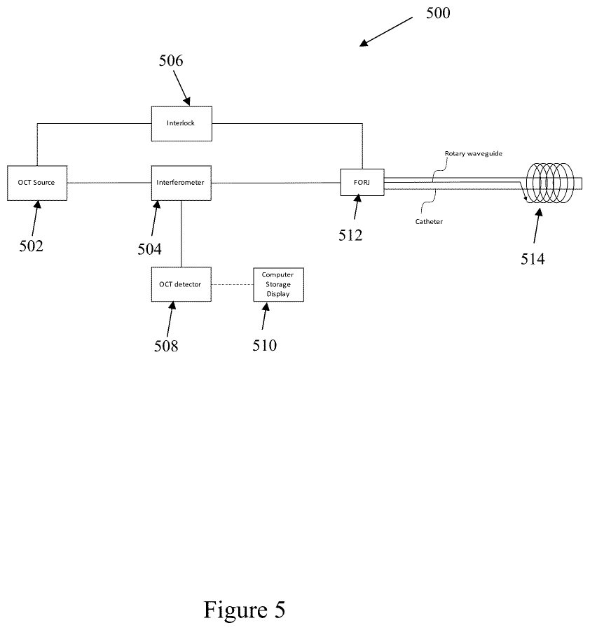

FIG. 5 shows a diagram of a third exemplary apparatus/system 500 which can utilize the exemplary method/process illustrated in FIG. 1 according to yet another exemplary embodiment of the present disclosure. The apparatus/system 500 can be an electromagnetic radiation system such as a catheter based imaging system. The exemplary apparatus/system 500 can include an optical coherence tomography (OCT) source 502, an interferometer 504, an interlock mechanism 506 including a safety circuit disclosed herein, an OCT detector 508, a computer storage display 510, and a fiber optic rotary joint (FORJ) 512 or a rotary junction that can facilitate the optical signals to pass from a stationary structure (e.g., the OCT source 502) to a rotating mechanism (e.g., a rotary waveguide) in a fiber optic system. The exemplary rotating mechanism can include a motor that can rotate in an endoscope so the B path is rotating through that endoscope. The interlock mechanism 506 can be configured to control the light transmission by either blocking or allowing the light transmission. The rotating mechanism can include, e.g., a spatial encoder that can encode the motion cycle of the motor. The interlock mechanism 506 can receive signals/pulses from the spatial encoder, and can control the light transmission based on the received signals/pulses, as described herein. In this exemplary embodiment, the motor can be configured to control the rotary waveguide to obtain trace of a light exposure 514.

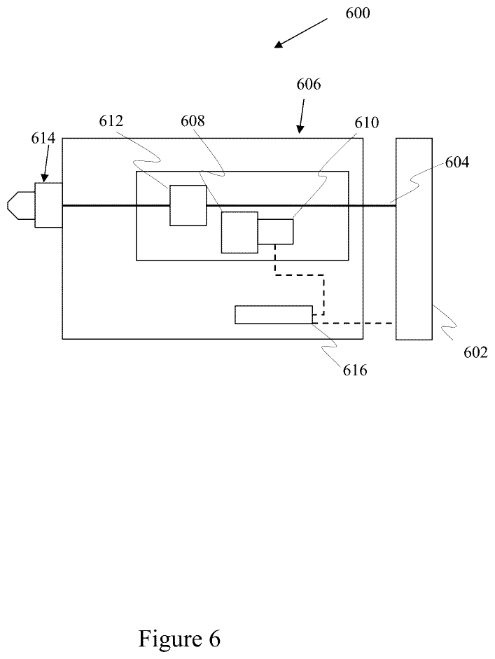

FIG. 6 shows a diagram an exemplary apparatus/system 600 having a safety feature for preventing or reducing the exposure from the electromagnetic radiation according to an exemplary embodiment of the present disclosure. The exemplary apparatus/system 600 can include a laser source 602, an optical relay 604 and a catheter interface unit 606. Inside the catheter interface unit 606, the system 600 can include a motor 608, a rotational encoder 610 associated with the motor 608, an aperture 612 through which the laser light can be transmitted, and an interlock mechanism 616 that can include a safety circuit. The exemplary apparatus/system 600 can also include a catheter connection interface 614 coupled to the catheter interface unit 606. The optical relay 604 can be configured to transmit the laser light from the laser source 602 to the catheter connection interface 614 via the aperture 612. The interlock mechanism 616 can be configured to receive rotational pulses or signals from the rotational encoder 610 that can detect the rotational speed/frequency of the motor 608.

The interlock mechanism 616 can control the light transmission based on the received signals/pulses, as described above. For example, if the rotational speed/frequency of the motor is smaller than a specified threshold frequency, such as 10, 20, . . . , 100000 RPM and everything in between, the interlock mechanism 616 can output a first signal to a controller of the system 600 to block transmission of laser light through the aperture 612, such that the laser output is reduced, e.g., to zero energy output. The outputted first signal can cause the controller of the exemplary apparatus/system 600 to completely or partially close a shutter to block the laser transmission through the aperture 612. In this exemplary way, the laser light would be prevented from arriving at a catheter disposed in the catheter connection interface 614, such that potential hazard or damage to tissues caused by the laser light can be reduced or prevented. Similarly, if the rotational speed/frequency of the motor is greater than a specified threshold frequency, such as 10, 20, . . . , 100000 RPM and everything in between, the interlock mechanism 616 can output a second signal to a controller of the exemplary apparatus/system 600 to allow transmission of laser light through the aperture 612, such that the laser output is satisfied for its intended purpose, such as, e.g., medical imaging. The outputted second signal can cause the controller of the exemplary apparatus/system 600 to completely or partially open the shutter so as to facilitate the laser transmission through the aperture 612. In this exemplary way, the laser light can arrive at the catheter disposed in the catheter connection interface 614, so that imaging by the laser light can be performed.

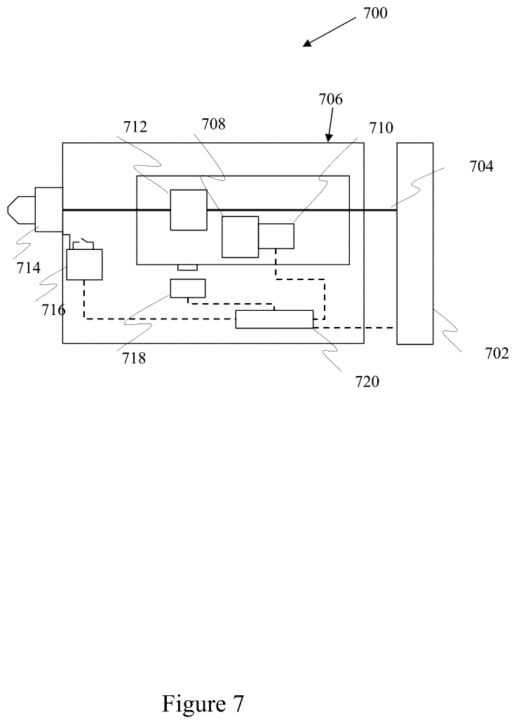

FIG. 7 shows a diagram of another exemplary apparatus/system 700 having a safety feature for preventing or reducing electromagnetic radiation exposure according to an exemplary embodiment of the present disclosure. The exemplary apparatus/system 700 can include a laser source 702, an optical relay 704, and a catheter interface unit (CIU) 706. Inside the catheter interface unit 706, the system 700 can include a motor 708, a rotational encoder 710 associated with the motor 708, an aperture 712 through which laser light can transmit, a catheter-lock switch 716, an opto-isolator home position sensor 718 that can detect a home position of the aperture 712, and a multi-input interlock mechanism/circuit 720 including a safety circuit. The exemplary apparatus/system 700 can also include a catheter connection interface 714 coupled to the catheter interface unit 706. The optical relay 704 can be configured to transmit laser light from the laser source 702 to the catheter connection interface 714 via the aperture 712.

For example, the interlock mechanism 720 can be configured to receive rotational pulses or signals from the rotational encoder 710 that can detect (i) the rotational speed/frequency of the motor 708, (ii) signals from the catheter-locked switch 716, whether a catheter is positioned in the catheter connection interface 714, and/or (iii) signals from the opto-isolator home position sensor 718 that can detect whether the aperture 712 is positioned at a home position. The interlock mechanism 720 can control the light transmission based on the received rotational pulses or signals from the rotational encoder 710, signals from the catheter-locked switch 716, and/or signals from the opto-isolator home position sensor 718. For example, if a catheter is installed in the catheter connection interface 714 (i.e., the catheter-locked switch 716 detects the catheter is positioned in the catheter connection interface 714) and the motor 708 is spinning the laser aperture 712 (e.g., the rotational speed/frequency of the motor 708 is greater than a specified threshold frequency, e.g., 10 RPM), the interlock mechanism 720 can output a signal to a controller of the exemplary apparatus/system 700 to facilitate the transmission of laser light through the laser aperture 712, such that the laser output is satisfied for its intended purpose, such as medical imaging.

The outputted signal can cause the controller of the exemplary apparatus/system 700 to completely or partially open the shutter to facilitate the laser transmission through the aperture 712. In this exemplary way, the laser light can arrive at the catheter disposed in the catheter connection interface 714, so that imaging or signal transmission/collection by the laser light can be performed. Similarly, if no catheter is installed in the catheter connection interface 714 (e.g., the catheter-locked switch 716 detects that no catheter is positioned in the catheter connection interface 714) and the CIU laser aperture 712 is at a safe distance from user-accessible catheter port (e.g., the opto-isolator home position sensor 718 detects the aperture 712 is positioned at a home position), the interlock mechanism 720 can output a signal to the controller of the exemplary apparatus/system 700 to facilitate the transmission of laser light through the laser aperture 712, such that the laser output is satisfied for its intended purpose, such as system diagnostics evaluation prior to medical imaging. The outputted signal can cause the controller of the exemplary apparatus/system 700 to completely or partially open the shutter to facilitate the laser transmission through the aperture 712. In this exemplary way, the laser light can arrive at a catheter connected to the catheter connection interface 714, so that imaging by the laser light can be performed.

In other exemplary situations, for example, when the rotational speed/frequency of the motor is smaller than the specified threshold frequency and/or the aperture 712 is not positioned at the home position, the interlock mechanism 720 can output a signal to the controller of the exemplary apparatus/system 700 to block the transmission of the laser light through the aperture 712, such that the laser output is reduced, e.g., to zero energy output. The outputted signal can cause the controller of the exemplary apparatus/system 700 to completely or partially close the shutter to block the laser transmission through the aperture 712. In this exemplary way, the laser light can be prevented from arriving at a catheter connection interface 714, such that potential hazard or damage to tissues caused by the laser light can be reduced or prevented.

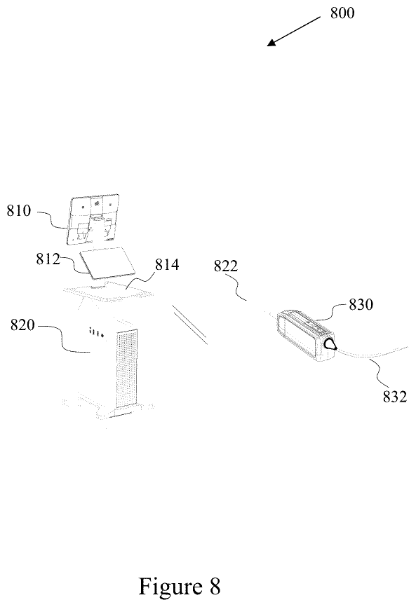

FIG. 8 shows a diagram an exemplary multimodal characterization apparatus/system 800 according to an exemplary embodiment of the present disclosure. The exemplary apparatus/system 800 can include a computer monitor 810, a console 820, and a catheter interface device 830 that can be connected to a laser. The system 800 can further comprise a technician monitor 812, a support plate or a tray 814, an electro-optical transmission cable 822 and a patient interfacing catheter 832. The catheter interface device 830 can interface with the console 820 via the electro-optical transmission cable 108 and the patient-interfacing catheter 112.

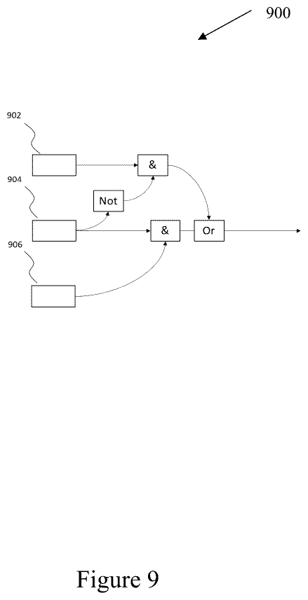

FIG. 9 shows a logic/block diagram of an exemplary system/apparatus 900 for preventing or reducing the exposure from the electromagnetic radiation according to an exemplary embodiment of the present disclosure. The exemplary apparatus/system 900 can be used to design an electromagnetic radiation system (e.g., the laser) to provide a hardware-mediated interlock/switch to prevent laser activation unless one of the following conditions is met: (1) no catheter is installed and the laser aperture is at a safe distance from a user-accessible port, which can be enforced by discrete logic (e.g., on a printed circuit board assembly (PCBA)) or (2) a catheter has been connected and an optical beam scanner (e.g., a rotational motor) is moving an optical beam location above a specific speed.

For example, when the interlock/switch is asserted, the laser emission can latch OFF in less than, for example, 2 micro seconds (e.g., according to the manufacturer's specification). As an additional fail-safe, the laser can be designed to not automatically re-enable laser emission when the interlock/switch is de-asserted. Instead, e.g., (1) interlock/switch can be de-asserted, and (2) a “Laser Emission ON” command can be issued (after interlock/switch de-asserted) via software interface over universal serial bus (USB) (e.g., StartScan( ) in API).

As shown in FIG. 9, a laser safe position signal can be provided into the interlock/switch by a laser-safe position switch 902 or sensor to determine whether the laser aperture is at a safe position. The laser-safe position switch or sensor can detect whether the laser aperture position is at a safe position, for example, by determining whether a linear motor that controls the laser aperture is at a safe position. This exemplary laser-safe position switch or sensor can be a transmission type photo-interrupter. A catheter installed signal can be provided into the interlock/switch by a catheter locked switch 904 (such as a momentary microswitch located in the catheter connector) to determine whether a catheter is installed. The catheter locked switch can detect whether a catheter has been installed at a catheter port of the laser system. As described herein, e.g., when the catheter locked switch detects that a catheter is not installed at the catheter port of the laser system and the laser-safe position switch or sensor detects that the laser aperture position is at a safe distance from user-accessible catheter port, the interlock/switch can be triggered to send a signal to the controller of the laser system to activate the laser or allow laser light to pass through a desired path for performing intended action.

In addition, as shown in FIG. 9, a rotation motor speed signal 906 can be provided into the interlock/switch by an incremental rotary encoder to determine the cycles or frequency of the rotation motor (e.g., and therefore the speed of translation of an scanning optical beam on a sample). The incremental rotary encoder can encode the cycles or frequency of the rotation motor as pulses and transmit the pulses to the interlock/switch. When the catheter locked switch detects that a catheter is installed at the catheter port of the laser system and the incremental rotary encoder detects that the rotation cycles or frequency of the rotation motor is greater than a predetermined cycle or frequency such as, e.g., 9 RPM or 0.15 Hz, the interlock/switch can be triggered to send a signal to the controller of the laser system to activate the laser or allow laser light to pass through a desired path for performing intended action.

In this exemplary embodiment, the interlock signal can be created by an opto-isolator that asserts or de-asserts the interlock at the laser. The interlock signal can cause the controller of the laser system to open or close a shutter to allow or block light transmission.

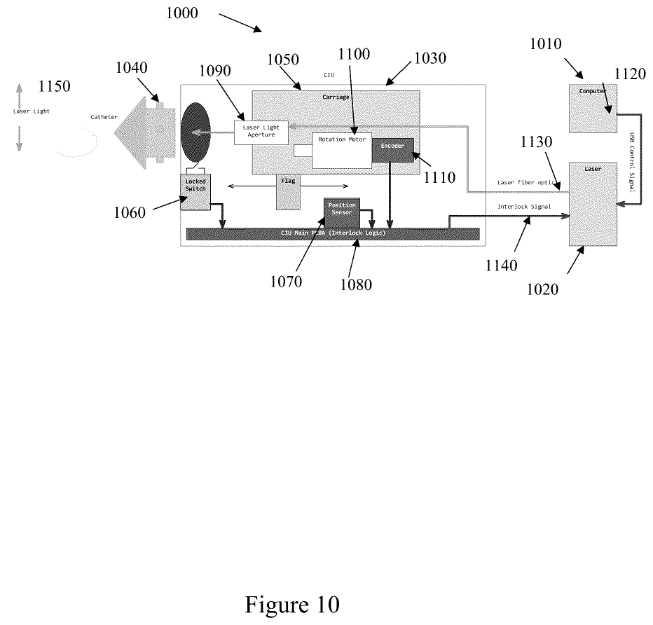

FIG. 10 shows a diagram of another imaging apparatus/system 1000 having the safety feature for preventing or reducing the exposure from the electromagnetic radiation according to an exemplary embodiment of the present disclosure. The exemplary apparatus/system 1000 can comprise a computer 1010, a laser 1020, a CIU main 1030, and a catheter mount port 1040. The CIU main 1030 can comprise a carriage 1050, a catheter locked switch 1060, a laser light aperture position sensor 1070, and a CIU main PCBA 1080 including an interlock/switch logic. The carriage 1050 can comprise a laser light aperture 1090, a rotation motor 1100, and an encoder 1110.

The exemplary computer 1010 can control the laser 1020, for example, by transmitting a USB control signal 1120 to the laser 1020. The laser 1020 can transmit laser light through a laser fiber optic 1130 to the laser light aperture 1090. The CIU main PCBA 1080—which can include an interlock/switch logic—can receive one or more of the following exemplary inputs: (i) rotation motor incremental encoder signals transmitted by the encoder 1110 that can detect and encode the rotation cycle or frequency of the rotation motor 1110; (ii) catheter insertion detection switch signals generated and transmitted by the catheter locked switch 1060 that can determine whether a catheter is installed at the catheter mount port 1040; and (iii) “laser safe position” optical switch signals generated and transmitted by the laser aperture position sensor 1070 that can determine whether the laser aperture 1090 is at a safe position.

After the CIU main PCBA 1080—which can include an interlock/switch logic—receives one or more of the above three inputs, the CIU main PCBA 1080—which can include an interlock/switch logic—can generate and transmit an interlock signal 1140 to the laser 1020 to control the laser light. For example, when the catheter locked switch 1060 detects that a catheter is not installed at the catheter mount port 1040 and the laser aperture position sensor 1070 detects that the laser light aperture 1090 is at a safe distance from user-accessible catheter port, the CIU main PCBA 1080 including an interlock/switch logic can be triggered to send a interlock signal 1140 to the controller of the laser system to activate the laser or facilitate the laser light to pass through a desired path for performing intended action. When the catheter locked switch 1060 detects that a catheter is installed at the catheter mount port 1040 and the encoder 1110 detects that the rotation cycles or frequency of the rotation motor 1100 is greater than a predetermined cycle or frequency such as, e.g., 9 RPM or 0.15 Hz, the CIU main PCBA 1080 including an interlock/switch logic can be triggered to send a interlock signal 1140 to the controller of the laser system to activate the laser or facilitate the laser light to pass through a desired path for performing intended action.

Exemplary embodiments of the present disclosure can include an interlock/switch safety circuit that can be configured to direct electromagnetic radiation based on an optical modality. The optical modality can be an optical coherence modality (e.g., OCT, NIRS, etc.). The controller can include a spatial encoder which can provide or direct a trigger signal which can be associated with the information. The device can include at least one optical component configured to move the location of an incident optical beam on a sample. The incident optical beam can be moved to take a measurement. The incident optical beam can be raster scanned to create an image. The incident optical beam can be used to be translationally and/or rotationally scanned to create an image. The incident optical beam can be translated to create an image (e.g., by translating the sample or by translating the optical beam).

According to certain exemplary embodiments of the present disclosure, an interlock/switch safety circuit can be provided that can be configured to direct electromagnetic radiation based on rates of motion from more than one device configured to control the location of an incident optical beam (e.g., a motor effectuating a rotation of the optical beam and a motor effectuating a translation of the optical beam). In some exemplary embodiments of the present disclosure, the interlock/switch safety circuit can be configured to direct electromagnetic radiation based on both an imaging probe's (e.g., a cardiac catheter imaging probe's) speed of rotation and speed of translation (e.g., during a pullback). In some exemplary embodiments of the present disclosure, the speed of optical beam movement by a first device configured to move the optical beam may cause a first determined amount of optical transmission (e.g., 10%, e.g., 20%, e.g., 30%, . . . e.g., 100%). In some exemplary embodiments of the present disclosure, the speed of optical beam movement by a second device configured to move the optical beam may cause a second determined amount of optical transmission (e.g., 10%, e.g., 20%, e.g., 30%, . . . e.g., 100%). %). According to further exemplary embodiments of the present disclosure, the combined speed of optical beam's movement by the first device and the second device can facilitate a determined amount of optical transmission. In additional exemplary embodiments of the present disclosure, an interlock/switch safety circuit that can be configured to direct electromagnetic radiation based on rates of motion from multiple devices configured to control the location of an incident optical beam and may vary control parameters based on a mode of operation (e.g., a user selectable mode of operation, a non-user selectable mode of operation using, for example, a bar/QR code located on a disposable item used or implemented for an exemplary procedure, etc.)

The exemplary embodiments of the present disclosure can facilitate a higher optical power on tissue after rotating at certain speed, which can be used for high-sensitivity optical coherence tomography (“OCT”) and/or near-infrared spectroscopy (“NIRS”) imaging systems Exemplary embodiments of the present disclosure can include an interlock/switch circuit that can facilitate safety, reduce accidental optical exposure to eyes/skin to remain in accordance with medical safety standards guidelines, reduce localized optical exposure to a tissue, and increase signals, signal noise ratio (SNR), OCT sensitivity (e.g., greater than about 90 dB sensitivity, e.g., greater than about 110 dB sensitivity). OCT systems can include, but not limited to, e.g., a splitter, a reference arm (e.g., mirror, rapid scanning optical delay line “RSOD”, non-reflective reference, etc.), an interferometer, a sample arm (which can include a non-biological or a biological sample or structure), an interferometric detector receiving the return signals/radiation from the sample and reference arms, interferes them, and generates an interfered signal, and a system/computer/controller configured to interpret the interfered signal, and generate information and/or images of the structure/sample based on the interpreted interfered signal.

Exemplary embodiments of the present disclosure can provide an electromagnetic radiation system/apparatus which can include an interlock/switch safety circuit. The disclosed electromagnetic radiation system can be configured to perform: optical diagnostics, such as imaging including OCT imaging, reflectance imaging, fluorescence imaging, Raman imaging, near-infrared imaging, spectroscopy including diffuse reflectance spectroscopy, fluorescence spectroscopy, Raman spectroscopy, and near-infrared spectroscopy, and/or optical therapeutics (e.g., marking, ablating). The exemplary electromagnetic radiation system/apparatus can be configured to receive multiple inputs and generate multiple outputs to control multiple optical sources, signal other software (e.g., trigger program), and signal other hardware (e.g., data acquisition (DAQ), pullback motor).

The exemplary electromagnetic radiation system/apparatus can be configured to have a minimum translation speed (e.g., 1 m/s, 50 m/s, 100 m/s, 1000 m/s, 10,000 m/s and everything in between or beyond) or rotation speed (e.g., 10 RPM, 100 RPM, 1000 RPM and everything in between or beyond) of a motor (e.g., a rotation motor) in order to accommodate: higher or lower laser energies, different tissue types (e.g., skin, eye, heart, brain), and different environmental conditions (e.g., temperature, humidity). In some exemplary embodiments, an optical beam on a sample, produced by the electromagnetic radiation system disclosed herein may have a maximum positional dwell time (e.g., pixel dwell time, e.g., voxel dwell time), for example 1 us (e.g., 1 ps, 1 ns, 1 μs, 1 ms, 10 s and everything in between or beyond).

The exemplary interlock/switch safety circuit can be configured to act as a switch to: connect/disconnect power to laser, output a signal (e.g., trigger) to the laser module to turn on light transmission, and/or open/close a shutter internal or external to the laser module. The exemplary interlock/switch safety circuit can be configured to act as a variable control to: control the position of an optical filter, control a variable transmission filter, control a duty cycle, and/or control the speed of laser repetition rate.

In some exemplary embodiments, the spatial encoder can output signals proportional to motor speed. In some exemplary embodiments, the “interlock”/safety switch can be hardware or software based, or both, preferably a hardware-based logic circuit—i.e., little or no software needed, thus lower regulatory burden on software class. In some embodiments, the “interlock”/safety switch can comprise multiple stages of interlocks for added safety (e.g., rotational speed, catheter locked, CIU carriage home position, temperature-sensitive, photo-sensitive). For example, the exemplary apparatus/systems described herein can include a temperature sensor that monitors the temperature of the rotation motor (e.g., heat can be generated when the rotation motor is rotating at a fast enough speed). When the monitored temperature is equal to or greater than a prespecified temperature threshold, the “interlock”/safety switch can be triggered to shut off the laser system or block transmission of the laser light.

In other exemplary embodiments, the “interlock”/safety switch can be configured for other hardware control, for example, linear pullback motor. For example, the linear pullback motor can be controlled by the interlock/safety switch to pull back the catheter where the light transmission is controlled. In other exemplary embodiments, the “interlock”/safety switch can be configured to receive rate of motion information from the linear pullback motor and this information can also be used to control optical transmission (e.g., in conjunction with a rotational motor speed). In some exemplary embodiments, an electromagnetic radiation system disclosed herein may have an optical power of greater than 1 mW (e.g., greater than. 01 mW, e.g., greater than 10 mW, e.g., greater than 10000 mW and everything in between or beyond). In some exemplary embodiments, an optical beam on a sample, produced by the electromagnetic radiation system disclosed herein can have a maximum irradiance, for example, of about 25 mW/μm² for e.g., about 500 mW optical beams with spot sizes greater than, e.g., about 5 μm. (e.g., less than an irradiance of about 100 mW/μm², e.g., less than an irradiance of about 1000 mW/μm², e.g., less than an irradiance of about 10000 mW/μm²).

As used in the present disclosure, a computer can include any device (which can have a processor) that can be configured to receive information, and, e.g., effectuate the generation or the output of the electromagnetic radiation if the rate of motion is greater than a predetermined rate based on the information. The computer can also include storage device(s) (e.g., memory, hard drive, RAM, ROM, removable storage devices, etc.), as well as network connectivity ports for receiving and transmitting data. The computer can also be or include a microprocessor, a logic circuit, etc.

According to another exemplary embodiment of the present disclosure, a method can be provided for controlling a generation or an output of an electromagnetic radiation. The exemplary method can comprise directing the electromagnetic radiation; controlling the device to move the electromagnetic radiation at a rate of motion using information regarding the rate of motion, receiving the information, and effectuating the generation or the output of the electromagnetic radiation if the rate of motion is greater than a predetermined rate based on the information.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the appended claims, and may include other examples that occur to those skilled in the art. Such other examples are intended to be within the scope of the appended claims if they have structural elements that do not differ from the literal language of the appended claims, or if they include equivalent structural elements with insubstantial differences from the literal language of the appended claims.

1. An apparatus for controlling a generation or an output of an electromagnetic radiation, comprising:
a device configured to direct the electromagnetic radiation;
a controller configured to control the device to move the electromagnetic radiation at a rate of motion, wherein at least one of the device or the controller provides information regarding the rate of motion; and
a computer configured to receive the information, and effectuate the generation or the output of the electromagnetic radiation if the rate of motion is greater than a predetermined rate based on the information.

2. The apparatus according to claim 1, further comprising a light source, wherein the computer is configured to control the generation or the output of the electromagnetic radiation by controlling the light source.

3. The apparatus according to claim 2, wherein the computer is configured to control at least one of a frequency or an intensity of the electromagnetic radiation based on the information.

4. The apparatus according to claim 1, wherein the device includes at least one of:
(i) at least one component configured for the electromagnetic radiation to allow to pass or at least partially prevent from passing in a controlled manner, wherein the computer is configured to control the generation or the output of the electromagnetic radiation by controlling the at least one component,
(ii) at least one optical component configured to move the at least one optical component or translate the location of an incident optical beam on a biological structure, or
(iii) a motor to direct the electromagnetic radiation.

5. The apparatus according to claim 4, wherein the at least one component includes a shutter or a light blocking configuration which is configured to be opened or closed by the computer.

6. (canceled)

7. The apparatus according to claim 4, wherein the movement of the at least one optical component is controlled by the controller.

8. The apparatus according to claim 1, wherein the controller is configured to be provided in a catheter system.

9. The apparatus according to claim 1, wherein the device is configured to direct the electromagnetic radiation based on an optical modality.

10. The apparatus according to claim 9, wherein the optical modality is an optical coherence tomography modality.

11. The apparatus according to claim 1, wherein the controller includes at least one of:
(i) a spatial encoder which provides a trigger signal which is associated with the information,
(ii) a catheter-lock switch which provides a trigger signal which is associated with the information, and wherein the catheter-lock switch is configured to determine an installation status of a catheter,
(iii) an aperture position switch which provides a trigger signal which is associated with the information, and wherein the aperture position switch is configured to determine a position of a light aperture, or
(iv) an interlock circuit that is configured to receive an input signal and transmit an output signal to the computer.

12. The apparatus according to claim 11, wherein the spatial encoder is configured to detect the rate of motion.

13-15. (canceled)

16. The apparatus according to claim 12, wherein the interlock circuit is configured to control an intensity of the output of the electromagnetic radiation.

17-18. (canceled)

19. The apparatus according to claim 4, wherein at least one of the device or the controller controls the incident optical beam to be raster scanned on the biological structure.

20. The apparatus according to claim 21, wherein at least one of the device or the controller controls the incident optical beam to be rotationally scanned on the biological structure.

21. A method for controlling a generation or an output of an electromagnetic radiation, comprising:
directing the electromagnetic radiation using a device;
controlling the device to move the electromagnetic radiation at a rate of motion using information regarding the rate of motion; and
receiving the information, and effectuating the generation or the output of the electromagnetic radiation if the rate of motion is greater than a predetermined rate based on the information.

22. The method according to claim 21, further comprising controlling the generation or the output of the electromagnetic radiation by controlling a light source.

23. The method according to claim 22, further comprising controlling at least one of a frequency or an intensity of the electromagnetic radiation based on the information.

24. The method according to claim 21, further comprising:
facilitating the electromagnetic radiation to pass or at least partially prevent from passing in a controlled manner; and
controlling the generation or the output of the electromagnetic radiation by controlling at least one component which facilitates the passing or at least partially preventing the passing of the electromagnetic radiation.

25. The method according to claim 24, wherein the at least one component includes a shutter or a light blocking configuration which is configured to be opened or closed by the computer.

26. The method according to claim 24, further comprising moving the at least one optical component.

38. The method according to claim 21, further comprising translating the location of an incident optical beam on a biological structure using at least one optical component.

27. The method according to claim 26, wherein the movement of the at least one optical component is controlled by a controller which controls the generation or the output of the electromagnetic radiation.

28. The method according to claim 21, wherein the controlling is performed by a controller which at least one of:
(i) is configured to be provided in a catheter system,
(ii) includes a catheter-lock switch providing a trigger signal which is associated with the information, further comprising determining an installation status of a catheter using the catheter-lock switch,
(iii) includes an aperture position switch providing a trigger signal which is associated with the information, further comprising determining a position of a light aperture using the aperture position switch, or
(iv) includes an interlock circuit that is configured to receive an input signal and transmit an output signal to the computer.

29. The method according to claim 21, wherein the directing of the electromagnetic radiation is based on an optical modality.

30. The method according to claim 29, wherein the optical modality is an optical coherence tomography modality.

31. (canceled)

32. The method according to claim 28, further comprising detecting the rate of motion using the spatial encoder.

33-35. (canceled)

36. The method according to claim 28, further comprising controlling an intensity of the output of the electromagnetic radiation using the interlock circuit.

37. The method according to claim 21, further comprising at least one of (i) directing the electromagnetic radiation using a motor, (ii) translating the location of an incident optical beam on a biological structure using at least one optical component, or (iii) controlling the incident optical beam to be rotationally scanned on the biological structure.

38. (canceled)

39. The method according to claim 37, wherein at least one of the device or the controller controls the incident optical beam to be raster scanned on the biological structure.

40. (canceled)

41. A non-transitory computer-readable medium for controlling a generation or an output of an electromagnetic radiation which includes instructions that, when executed on a computer configuration, cause the computer configuration to perform procedures comprising:
controlling the device to direct the electromagnetic radiation using a device, and to move the electromagnetic radiation at a rate of motion using information regarding the rate of motion; and
receiving the information, and effectuating the generation or the output of the electromagnetic radiation if the rate of motion is greater than a predetermined rate based on the information.
